(19) United States
(12) Patent Application Publication
(10) Pub. No.: US 2014/0135673 A1
(43) Pub. Date: May 15, 2014

(54) LUMBAR SUPPORT SYSTEM

(71) Applicants: TODD J. COHEN, PORT WASHINGTON, NY (US); BRITTANY A. COHEN, PORT WASHINGTON, NY (US)

(72) Inventors: TODD J. COHEN, PORT WASHINGTON, NY (US); BRITTANY A. COHEN, PORT WASHINGTON, NY (US)

(21) Appl. No.: 14/054,325

(22) Filed: Oct. 15, 2013

Related U.S. Application Data
(60) Provisional application No. 61/712,966, filed on Oct. 12, 2012.

(51) Int. Cl. A61F 5/02

(52) U.S. Cl. CPC A61F 5/028
USPC 602/19; 29/428

(57) ABSTRACT

A lumbar support system includes a support element, incorporated in or used in conjunction with a radiation protective apron or another article of clothing. The support element may be held in place in a user or wearer's lumbosacral region using a strap or belt-like member that is connected thereto or wrapped around the apron or other article of clothing, to apply a force external to a user or wearer to enhance lumbosacral support. The support element may be movably mounted on a bracket or be otherwise adjustable to allow its position to be shifted if desired.

### CROSS-REFERENCE TO RELATED APPLICATION

This application is based upon and claims the priority of co-pending, commonly owned U.S. Provisional Patent Application Ser. No. 61/712,966, filed Oct. 12, 2012, which is incorporated herein by reference in its entirety.

### FIELD OF INVENTION

This invention relates to a support system to provide lumbar support for radiation protective garments. More particularly, this invention relates to a system for providing lumbar support to personnel who are required to wear radiation protection equipment for extended periods of time.

### BACKGROUND OF THE INVENTION

Back pain is a common medical problem for many people. In many cases, a person's occupation is a contributing factor to their pain. Medical personnel who work in radiology, for example, are required to wear heavy, radiation protective or resistant clothing throughout their workday. The additional weight of this clothing, which often includes lead, lead-based materials, or other metals or materials to shield a user from radiation, can compress spaces between vertebral discs and cause muscles to tighten, which results in a variety of ailments, including, but not limited to, general discomfort, lower back pain, radiculopathy and sciatica, among others.

Accordingly, it would be desirable to a system to provide a support system to minimize or avoid the above and other problems.

### OBJECTS OF THE INVENTION

It is an object of the invention to provide a lumbar support and system for radiation protective garments that provides enhanced comfort to the lumbosacral area and minimizes back pain or discomfort.

It is also an object of the invention to provide a lumbar support and system where a lumbar support is held in place by a strap member connected to it and attached to or wrapped around an apron or another article of clothing.

It is a further object of the invention to provide a lumbar support and system for use in conjunction with radiation protective clothing or gear such as an apron, smock, jacket, vest, skirt, or any other wearable garment or clothing design, wherein a lumbar support is an integral part of the clothing or gear.

It is a yet further object of the invention to provide a lumbar support and system for use in conjunction with radiation protective clothing or gear wherein the lumbar support is capable of being attached and/or incorporated into the clothing or gear.

It is a yet further object of the invention to provide a system for modifying standard radiation protective gear or a radiation protective garment to provide lumbosacral support to a wearer.

It is a yet further object of the invention to provide a system for modifying standard radiation protective gear or a radiation protective garment to provide lumbosacral support to a wearer wherein the radiation protective gear or garment is an apron, vest, skirt, jacket, smock, or other wearable garment or clothing design.

It is a yet further object of the invention to provide a system for modifying standard radiation protective gear or a radiation protective garment to provide lumbosacral support to a wearer, which system comprises:

a mechanism or means capable of adapting a lumbosacral support to the inside, outside, or interior of the radiation protective gear or garment;

a mechanism or means capable of adjusting the lumbosacral support to the spinal configuration of a user or wearer; and

a mechanism or means for applying an external force to enhance lumbosacral support.

It is a yet further object of the invention to provide a system for modifying standard radiation protective gear or a radiation protective garment to provide lumbosacral support to a wearer, which system comprises a fastening mechanism, an internal or external belt system, or a combination of a fastening mechanism and an internal or external belt system to apply force to enhance lumbosacral support.

It is a yet further object of the invention to provide a method of modifying standard radiation protective gear or a radiation protective garment, which comprises:

adapting lumbosacral support to the inside, outside, or interior of the radiation protective gear or garment;

providing a mechanism or means for adjusting the support to the spinal configuration of a user or wearer; and

providing a mechanism or means for applying an internal or external force via a fastening mechanism, a belt system, or a combination of a fastening mechanism and a belt system to enhance lumbosacral support.

It is a yet further object of the invention to provide a pad, cushion, or supportive material to be positioned on and/or in contact with a wearer's lumbosacral region, which pad, cushion, or supportive material can be positioned on or in radiation protective gear or a radiation protective garment and which pad, cushion, or supportive material is capable of alleviating back pain and/or discomfort.

It is a yet further object of the invention to provide an improved radiation protective gear or garment wherein the improvement comprises a pad, cushion, or supportive material capable of being positioned on and/or in contact with a wearer's lumbosacral region, which pad, cushion, or supportive material is capable of alleviating back pain, discomfort, or both back pain and discomfort.

It is a yet further object of the invention to provide an improved radiation protective gear or garment which also comprises a mechanism or means capable of adjusting a lumbosacral support to the spinal configuration of a user or wearer.

It is a yet further object of the invention to provide an improved radiation protective gear or garment wherein an orthotic such as a pad, cushion, or supportive material can be adjusted vertically or horizontally in relation to a wearer to position the orthotic at the wearer's lumbosacral region.

It is a yet further object of the invention to provide an improved radiation protective gear or garment which also comprises a mechanism or means for applying an internal or external force via a fastening mechanism or system, a belt system, or a combination of a fastening mechanism or system and a belt system to enhance lumbosacral support.

It is a yet further object of the invention to provide an improved radiation protective gear or garment wherein the radiation protective gear or garment is an apron, vest, skirt, jacket, smock, or other wearable garment or clothing design.

It is a yet further object of the invention to provide an improved radiation protective gear or garment wherein a pad, cushion, or supportive material forms part of a belt-like member having a rear section that permanently or removably attaches to a rear interior or exterior surface of the gear or garment.

It is a yet further object of the invention to provide an improved radiation protective gear or garment wherein a pad, cushion, or supportive material can be adjusted vertically or horizontally on a belt-like member in relation to a wearer to position the pad, cushion, or supportive material at the wearer's lumbosacral region.

It is a yet further object of the invention to provide an improved radiation protective gear or garment wherein a wearer can adjust one or more wires that extend from a front or lateral portion of a belt-like member or system through the belt-like member or system to the pad, cushion, or supportive material.

It is a yet further object of the invention to provide an improved radiation protective gear or garment which comprises two or more vertical splines extending from a belt-like member for stability.

It is a yet further object of the invention to provide an improved radiation protective gear or garment wherein a pad, cushion, or supportive material comprises material capable of retaining heat or cold.

It is a yet further object of the invention to provide a system for protecting a user or wearer having a lumbosacral region from radiation, which comprises:

radiation protective gear or a radiation protective garment capable of being worn by the user or wearer; and

a pad, cushion, or supportive material within or attached to the radiation protective gear or garment,

wherein the pad, cushion, or supportive material is capable of being positioned on and/or in contact with the user or wearer's lumbosacral region and the pad, cushion, or supportive material is capable of alleviating back pain and/or discomfort.

It is a yet further object of the invention to provide a system for protecting a user or wearer having a lumbosacral region from radiation which also comprises a mechanism or means capable of adjusting the lumbosacral support to comport to the spinal configuration of the user or wearer.

It is a yet further object of the invention to provide a system for protecting a user or wearer having a lumbosacral region from radiation which also comprises a mechanism or means for applying an external force via a fastening mechanism or system, a belt system, or a combination of a fastening mechanism or system and a belt system to enhance lumbosacral support.

It is a yet further object of the invention to provide a system for protecting a user or wearer having a lumbosacral region from radiation wherein the radiation protective gear or garment is an apron, vest, skirt, jacket, smock, or other wearable garment or clothing design.

It is a yet further object of the invention to provide a system for protecting a user or wearer having a lumbosacral region from radiation which also comprises a mechanism or means for applying an external force via a fastening mechanism or a belt system internal or external to the radiation gear or garment to enhance lumbosacral support.

It is a yet further object of the invention to provide a system for protecting a user or wearer having a lumbosacral region from radiation wherein a pad, cushion, or supportive material forms part of a belt-like member or system having a rear section that permanently or removably attaches to a rear interior surface of radiation protection gear or a radiation protective garment.

It is a yet further object of the invention to provide a system for protecting a user or wearer having a lumbosacral region from radiation wherein a pad, cushion, or supportive material can be adjusted vertically or horizontally on a belt-like member or system in relation to the user or wearer to position the pad, cushion, or supportive material adjacent to the user or wearer's lumbosacral region.

It is a yet further object of the invention to provide a system for protecting a user or wearer having a lumbosacral region from radiation wherein a wearer can adjust one or more wires that extend from a front or lateral portion of a belt-like member or system through the belt-like member or system to a pad, cushion, or supportive material.

It is a yet further object of the invention to provide a system for protecting a user or wearer having a lumbosacral region from radiation, wherein a belt-like member or system comprises two or more vertical splines extending from the belt-like member or system for stability.

It is a yet further object of the invention to provide a system for protecting a user or wearer having a lumbosacral region from radiation wherein a pad, cushion, or supportive material comprises a material capable of retaining heat or cold.

These and other objects of the invention will become more apparent in the description below which refers to the accompanying drawings.

### SUMMARY OF THE INVENTION

In accordance with the invention, a lumbar support system alleviates the back pain and discomfort that medical personnel often experience as a result of the weight of radiation protective clothing. It has been found that when a orthotic such as pad, cushion, or other supportive material or device is positioned firmly in a wearer's lumbosacral region, preferably with a strap or belt member that extends to the wearer's front side, the wearer does not experience the back pain and/or discomfort that is typical when wearing radiation protective garments.

In one embodiment of the invention, an orthotic or prosthetic device such as a pad, cushion, or other supportive material or device is incorporated into radiation protective gear or a radiation protective garment during manufacture. The pad, cushion, or other supportive material or device can be sewn, glued, snapped, buttoned, or otherwise affixed within or to the inside of gear or a garment at a position where the pad, cushion, or other supportive material or device will contact a user or wearer's lumbosacral region. Optionally, there could be a pocket or other cavity in the gear or garment where a pad, cushion, or other supportive material or device could be secured. Either that gear or garment or a companion gear or garment, for example, an apron or a vest, will have at least one strap or belt that reaches around to the wearer's front and can be cinched to cause the pad, cushion, or other supportive material or device to press against the wearer's lumbar region.

In another embodiment of the invention, an orthotic such as a pad, cushion, or other supportive material or device is capable of being attached to radiation protective gear or a radiation protective garment that is “retrofitted” to accept it. The pad, cushion, or other supportive material or device can be sewn, glued, snapped, buttoned, or otherwise affixed to the inside of a radiation protective garment, either directly to the garment itself or to support members that were attached to the garment. The pad, cushion, or other supportive material or device should be attached in a position where the pad, cushion, or other supportive material or device will contact the wearer's lumbosacral region. Either that garment or a companion garment, for example, an apron and then a vest, will have at least one strap or belt that reaches around to the wearer's front and can be cinched to cause the pad, cushion, or other supportive material or device to press against the wearer's lumboscaral region. Optionally the pad, cushion, or other supportive material or device may have such a strap or belt itself. Also, optionally the pad, cushion, or other supportive material or device can be sewn, glued, snapped, buttoned, or otherwise affixed to the outside of a radiation protective garment, either directly to the garment itself or to support members that were attached to the garment.

In another embodiment of the invention, the lumbar support system comprises an orthotic such as a pad, cushion, or other supportive material or device that has an attached strap or belt system that is intended to reach around to a wearer's front side. The orthotic such as a pad, cushion, or other supportive material or device will optionally have a front, resilient surface that is intended to face a wearer's lumbosacral region and a more rigid rear surface that is intended to engage or support a strap or belt. The strap or belt may be affixed to the rear surface or one section of the strap or belt could be attached to one side of the rear surface, and another part of the strap or belt could be attached to the other side of the rear surface. Optionally, the strap or belt could engage buckles or loops on the rear surface, or ends of portions of the strap or belt could be affixed to loops or buckles on the rear surface or on lateral surfaces of the orthotic such as a pad, cushion, or other supportive material or device.

In one embodiment of the invention, a system is capable of modifying standard radiation protective gear or a radiation protective garment to provide lumbosacral support to a wearer.

In another embodiment of a system of the invention, the radiation protective gear or garment is an apron, vest, skirt, jacket, smock, or other wearable garment or clothing design.

In another embodiment of a system of the invention, the system comprises:

a mechanism or means capable of adapting a lumbosacral support to the inside or interior of radiation protective gear or a radiation protective garment;

a mechanism or means capable of adjusting the lumbosacral support to the spinal configuration of a user or wearer; and

a mechanism or means for applying a force external to the user or wearer to enhance lumbosacral support.

In another embodiment of a system of the invention, the mechanism or means to apply force external to a user or wearer is a fastening mechanism, a belt system, or a combination of a fastening mechanism and a belt system.

In another embodiment of a system of the invention, the belt system is external to, internal to, or within radiation protective gear or a radiation protective garment.

In another embodiment of the invention, a method of modifying standard radiation protective gear or a garment comprises:

adapting a lumbosacral support to the inside or interior of the radiation protective gear or garment;

providing a mechanism or means for adjusting the support to the spinal configuration of a user or wearer; and

providing a mechanism or means for applying force external to the user or wearer to enhance lumbosacral support.

In another embodiment of a method of the invention, the mechanism to apply force external to the user or wearer is a fastening mechanism or system, a belt system, or a combination of a fastening mechanism or system and a belt system.

In another embodiment of a method of the invention, the belt system is external to, internal to, or within radiation gear or a radiation garment.

In another embodiment of the invention, in an orthotic such as a pad, cushion, or supportive material or device to be positioned on and/or in contact with a user or wearer's lumbosacral region, the orthotic such as a pad, cushion, or supportive material or device can be positioned on or in radiation protective gear or a radiation protective garment and the orthotic such as a pad, cushion, or supportive material is capable of alleviating back pain and/or discomfort.

In another embodiment of the invention, in an improved radiation protective gear or garment, the improvement comprises an orthotic such as a pad, cushion, or supportive material capable of being positioned on and/or in contact with a user or wearer's lumbosacral region, which orthotic is capable of alleviating back pain, discomfort, or both back pain and discomfort.

In another embodiment of an improved protective gear or garment according to the invention, the gear or garment also comprises a mechanism or means capable of adjusting the lumbosacral support to comport to the spinal configuration of a user or wearer.

In another embodiment of an improved radiation protective gear or garment according to the invention, an orthotic such as a pad, cushion, or supportive material or device can be adjusted vertically or horizontally in relation to a user or wearer to position the orthotic at or adjacent to the user or wearer's lumbosacral region.

In another embodiment of an improved radiation protective gear or garment according to the invention, the gear or garment also comprises a mechanism or means for applying a force external to the user or wearer via a fastening mechanism or system, a belt system, or a combination of a fastening mechanism or system and a belt system to enhance lumbosacral support.

In another embodiment of an improved radiation protective gear or garment according to the invention, the belt system is external to, internal to, or within radiation gear or a radiation garment.

In another embodiment of an improved protective gear or garment according to the invention, the radiation protective gear or garment is an apron, vest, skirt, jacket, smock, or other wearable garment or clothing design.

In another embodiment of an improved protective gear or garment according to the invention, an orthotic such as a pad, cushion, or supportive material or device forms part of a belt-like member or system having a rear section that permanently or removably attaches to a rear interior surface of the gear or garment.

In another embodiment of an improved protective gear or garment according to the invention, the gear or garment comprises an orthotic such as a pad, cushion, or supportive material or device that can be adjusted vertically or horizontally on the belt-like member or system in relation to a user or wearer to position the orthotic at or adjacent to the user or wearer's lumbosacral region.

In another embodiment of an improved protective gear or garment according to the invention, a user or wearer can adjust one or more wires that extend from a front or lateral portion of the belt-like member through the belt-like member to an orthotic such as a pad, cushion, or supportive material or device.

In another embodiment of an improved protective gear or garment according to the invention, the radiation protective gear or garment comprises two or more vertical splines extending from a belt-like member for stability.

In another embodiment of an improved protective gear or garment according to the invention, an orthotic such as a pad, cushion, or supportive material comprises material capable of retaining heat or cold.

In another embodiment of the invention, a system for protecting a user or wearer having a lumbosacral region from radiation, comprises:

radiation protective gear or a radiation protective garment capable of being worn by the user or wearer; and

an orthotic such as a pad, cushion, or supportive material or device within or attached to the radiation protective gear or garment,

wherein the orthotic is capable of being positioned on and/or in contact with the user or wearer's lumbosacral region and the orthotic is capable of alleviating back pain and/or discomfort.

In another embodiment of a system of the invention for protecting a user or wearer having a lumbosacral region from radiation, the system also comprises a mechanism capable of adjusting the lumbosacral support to comport to the spinal configuration of a user or wearer.

In another embodiment of a system of the invention for protecting a user or wearer having a lumbosacral region from radiation, the system also comprises a mechanism or means for applying a force external to the user or wearer via a fastening mechanism or system, a belt system, or a combination of a fastening mechanism or system and a belt system to enhance lumbosacral support.

In another embodiment of a system of the invention for protecting a user or wearer having a lumbosacral region from radiation, the belt system is external to, internal to, or within radiation gear or a radiation garment.

In another embodiment of a system of the invention for protecting a user or wearer having a lumbosacral region from radiation, the system comprises radiation protective gear or a garment that is an apron, vest, skirt, jacket, smock, or other wearable garment or clothing design.

In another embodiment of a system of the invention for protecting a user or wearer having a lumbosacral region from radiation, the system also comprises a mechanism or means for applying an external force via a fastening mechanism or system, a belt system, or a combination of a fastening mechanism or system and a belt system to enhance lumbosacral support.

In another embodiment of a system of the invention for protecting a user or wearer having a lumbosacral region from radiation, an orthotic such as a pad, cushion, or supportive material or device forms part of a belt-like member or system having a rear section that permanently or removably attaches to a rear interior surface of the gear or garment.

In another embodiment of a system of the invention for protecting a user or wearer having a lumbosacral region from radiation, an orthotic such as a pad, cushion, or supportive material or device can be adjusted vertically or horizontally on the belt-like member or system in relation to the wearer to position the orthotic at or adjacent to the user or wearer's lumbosacral region.

In another embodiment of a system of the invention for protecting a user or wearer having a lumbosacral region from radiation, the user or wearer can adjust one or more wires that extend from a front or lateral portion of the belt-like member through the belt-like member to an orthotic such as a pad, cushion, or supportive material or device.

In another embodiment of a system of the invention for protecting a user or wearer having a lumbosacral region from radiation, the system comprises two or more vertical splines extending from a belt-like member for stability.

In another embodiment of a system of the invention for protecting a user or wearer having a lumbosacral region from radiation, an orthotic such as a pad, cushion, or supportive material or device comprises material capable of retaining heat or cold.

In another embodiment of the invention, a lumbar support system comprises an orthotic such as a pad, cushion, or other supportive material or device and an attached strap or belt system that is intended to reach around to a user or wearer's front side.

In another embodiment of a lumbar support system of the invention, an orthotic such as a pad, cushion, or other supportive material or device will optionally have a front, resilient surface that is intended to face a user or wearer's lumbosacral region and a more rigid rear surface that is intended to engage or support a strap or belt.

In another embodiment of a lumbar support system of the invention, a strap or belt may be affixed to the rear surface of an orthotic, or one section of the strap or belt could be attached to one side of the front or rear surface and another part of the strap or belt could be attached to the other side of the front or rear surface.

In another embodiment of a lumbar support of the invention, a strap or belt could engage buckles or loops on the front or rear surface of an orthotic, or ends of portions of the strap or belt could be affixed to loops or buckles on the front or rear surface or on lateral surfaces of the orthotic.

In another embodiment of a lumber support of the invention, a strap or belt and orthotic comprise a belt-like member and a user or wearer can adjust one or more wires that extend from a front or lateral portion of the belt-like member through the belt-like member to the orthotic.

In another embodiment of the invention, an orthotic comprises a battery operated heating, massage, or vibrating device.

Before at least one embodiment of the invention is explained in detail, it is to be understood that the invention is not necessarily limited in its application to the details of construction and the arrangement of the components and/or methods set forth in the following description and/or illustrated in the drawings. The invention is capable of other embodiments or of being practiced or carried out in various ways.

### BRIEF DESCRIPTION OF THE DRAWINGS

FIG. 1 is an illustration of a front of a lumbar support system in accordance with an embodiment of the present invention;

FIG. 2 is an illustration of a cross-sectional view of the lumbar support system of FIG. 1;

FIGS. 3A to 3C are illustrations of front, lateral, and rear views, respectively, of a prosthetic support useful according to the invention;

FIGS. 4A and 4B are illustrations of another embodiment of the invention;

FIG. 5 is an illustration of a further embodiment of the invention; and

FIGS. 6 to 9 are illustrations of additional embodiments of the invention.

### DETAILED DESCRIPTION OF THE INVENTION

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

The lumbar support system of the present application is intended for use in conjunction with radiation protective gear or a radiation protective garment. The radiation protective gear or garment may be any conventional apron, smock, jacket, vest, skirt, or any other wearable garment or clothing design of radiation protective clothing. In one embodiment, a lumbar support system includes an orthotic such as a pad, cushion, or support material or device positioned on the inner surface of a skirt part of a protective apron, or other protective garment, adjacent to a user's back.

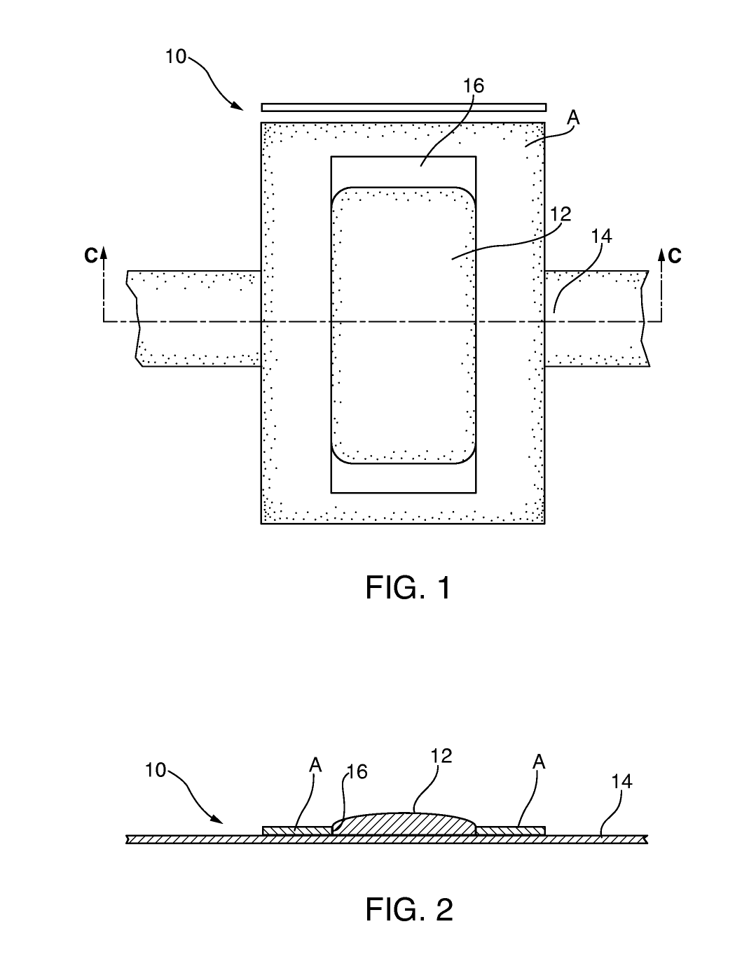

FIG. 1 illustrates a “front” view of a rear section 10 of a lumbar support system according to the invention. The “front” of an orthotic support element 12 is positioned adjacent to a user's back (not shown) during use. The front surface of orthotic support element 12 is preferably shaped to compliment the lumbosacral area or region of the user or wearer's back (not shown) and to provide lumbar support. Outside of a radiation protective garment A and parallel to orthotic support element 12 is an adjustable belt or strap 14, which when tightened will apply a force vector through orthotic support element 12 to the user or wearer's lumbosacral region. Orthotic support element 12 is removably attached to a section of radiation protection apron garment A.

FIG. 2 illustrates a cross-sectional view of the lumbar support system section 10 along the line C-C in FIG. 1. Orthotic support element 12 is fastened or otherwise connected to the inner surface of the radiation protective garment A. The positioning of orthotic support element 12 may be varied by a variety of means since orthotic support element 12 is preferably coupled to the radiation protective garment A by attachment member 16. Attachment member 16 may be a synthetic adjustable bracket as is discussed below, a hook and loop type connector, or other suitable connector. Orthotic support element 12 is preferably held in place by strap or belt 14 that may be tightened around the user or wearer's waist to apply focal lumbar support. The shape of orthotic support element 12 is preferably formed to provide lumbar support to a wide variety of users. Alternatively, the shape of orthotic support element 12 may be adjustable, that is, moldable or otherwise reshaped to a suitable contour for a particular user.

In one embodiment, orthotic support element 12 is positioned in or on the skirt of the apron or other item of clothing at the lumbosacral level. In the embodiment illustrated in FIG. 1, for example, orthotic support 12 may be mounted on bracket 16, which is provided on the inner surface of apron A or another article of clothing. Orthotic support element 12 is preferably movably mounted on bracket 16 such that the height of orthotic support element 12 is adjustable. Bracket 16 may include tracks on which the orthotic support element is slidable or movable up and down, for example. This allows the user to properly align the orthotic support element 12 to provide proper lumbar support. In this embodiment, an outer strap 14 may be provided to apply force over the material of the apron A, or other item of clothing, to translate the appropriate support to the lumbosacral area. Alternatively, the strap 14 may be directly connected to the bracket 16 and may be worn inside or outside the apron A. As noted above, a hook and loop type connector may be used as element 16 to allow repositioning of the support as well. Any connector that allows adjustment of the position of the support may be used.

In another embodiment, as mentioned above, orthotic support element 12 can be formed from a flexible material that is pliable to automatically adjust to the contour of a user's lumbosacral area. Orthotic support element 12 may also be moldable or otherwise reshaped into a desired contour. In this embodiment, however, the material of orthotic support element 12 should have sufficient tensile strength such that force is transferred to the lumbosacral area from strap 14.

Orthotic support element 12 of this embodiment may be provided on bracket 16 as described above or simply provided on strap 14, as desired. Strap 14 may be provided with orthotic support element 12 and bracket 16, or it may be wrapped around apron A or another article of clothing which includes orthotic support 12 and a bracket 16.

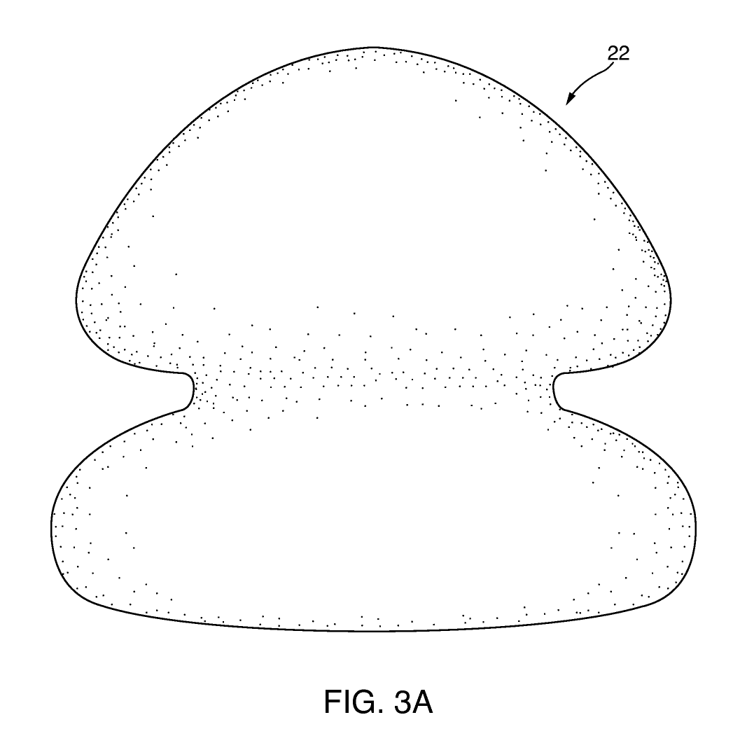

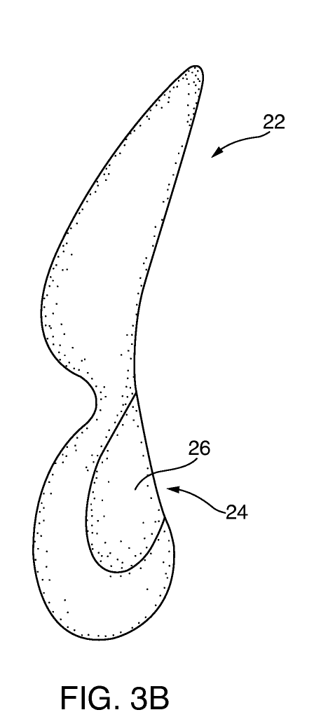

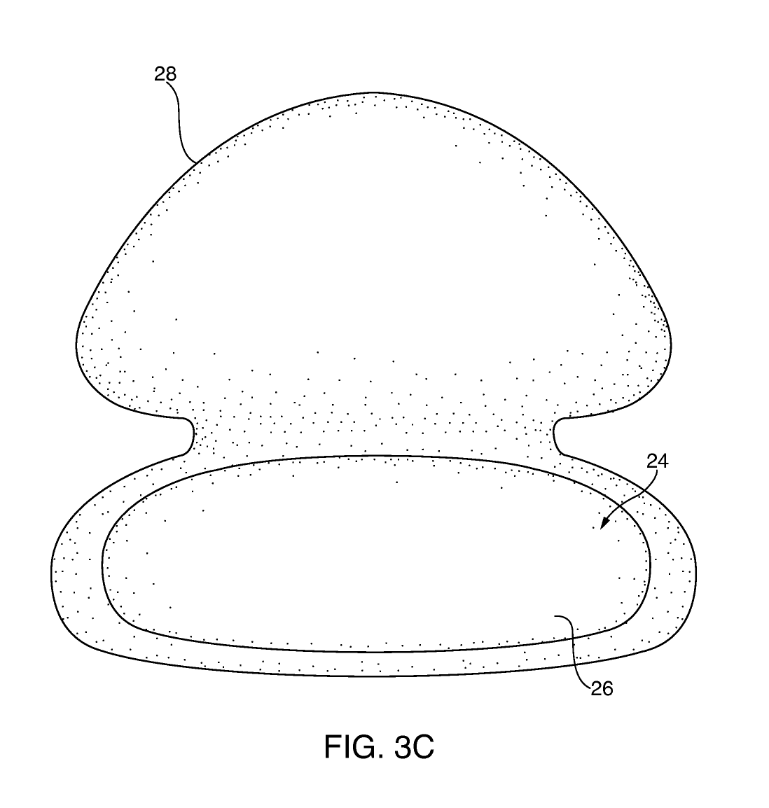

FIGS. 3A to 3C represent views of a orthotic support device useful according to the invention. FIG. 3A is a front view of orthotic support 22, which comprises a firm but flexible or moldable, optionally wipable, cleanable, or sterilizable, material that can be shaped to fit a user or wearer's lumbosacral region. Suitable materials for the substrate include, but are not limited to, known polymeric foams and polymers.

FIG. 3B is a lateral view of the orthotic support shown in FIG. 3A, and FIG. 3C is a rear view. Area 24 represents an opening where substrate 26 can be seen, most of which is covered by cover 28. Cover 28 preferably comprises a flexible, wipable, cleanable, or sterilizable material such as any of many known polyvinyl, polyethylene, polyacetate, or polyurethane materials or foams.

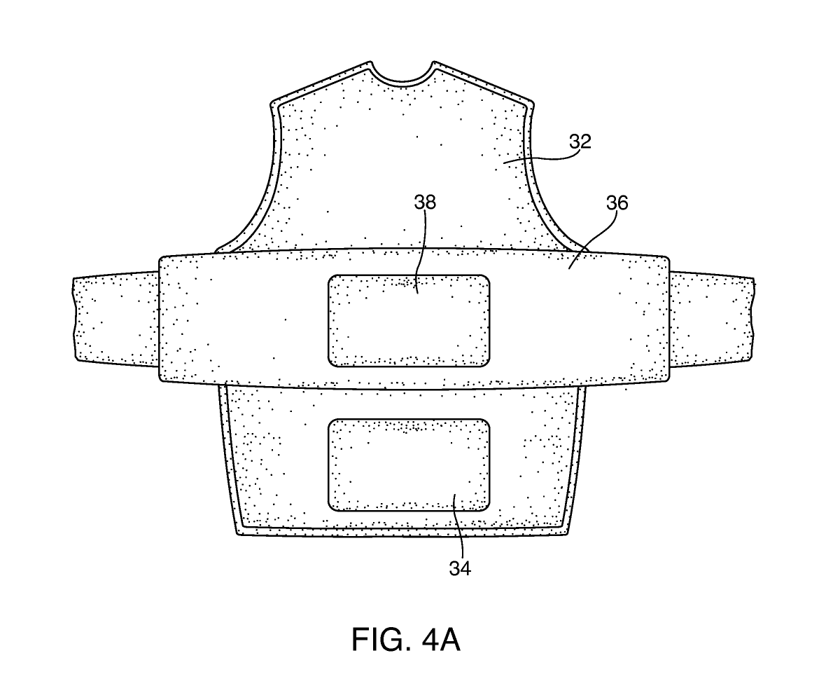

FIGS. 4A and 4B represent an embodiment of the invention as applied to a radiation protective vest 32. A VELCRO fastener patch 34 is attached to the lower inside surface of vest 32. A belt or strap 36 with a complimentary VELCRO surface 38 is shown positioned above patch 34, with the rear surface 40 of strap 36 facing up. For use, strap 36 is rotated so that surface 38 interlocks or meshes with patch 34. Strap 36 has ends (not shown) that can be buckled or otherwise connected around a user's waist.

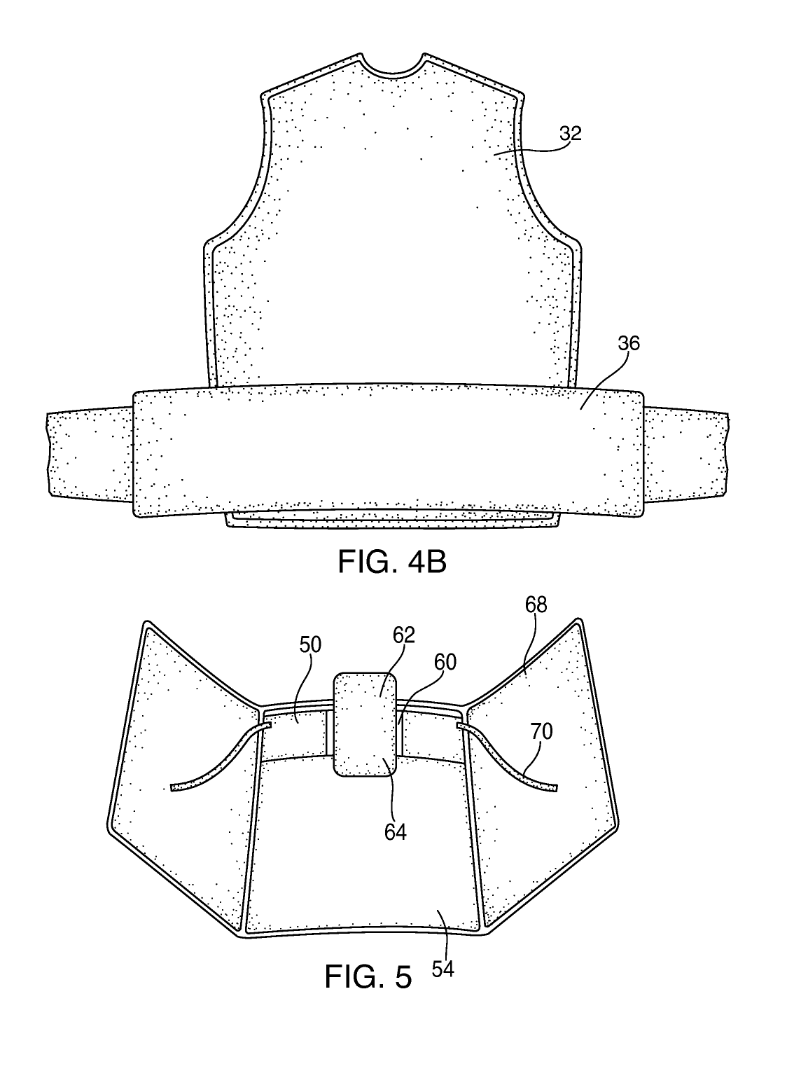

Another embodiment of the invention is shown in FIG. 5, where a belt or strap 50 is attached to the inner top surface of an apron 54. An attachment section 60 may comprise, for example, a VELCRO patch or surface. A orthotic pad or cushion 62 has a complimentary surface 64 that engages or interlocks with attachment section 60.

Apron 54 wraps around a user (not shown) so that an apron upper section 68 is at or above the user's waist. Upper section 68 and/or apron 54 will have appropriate closure members, such as, for example, buttons, ties, snaps, VELCRO sections, or the like, to keep apron 54 snugly bound around the user's waist area. Strap or belts 70 may assist in holding apron 54 up.

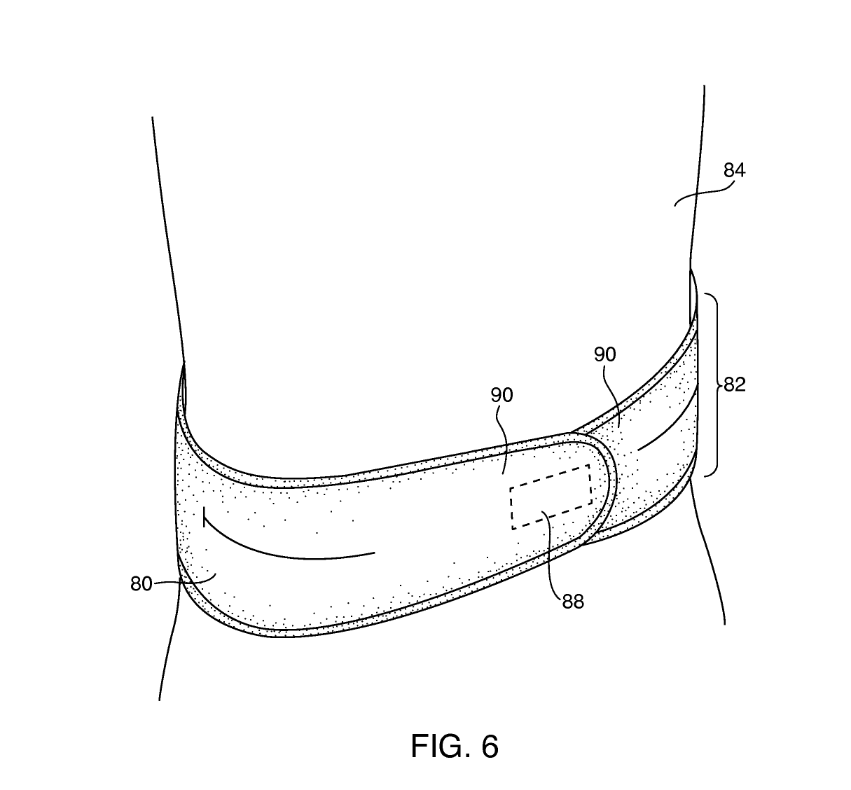

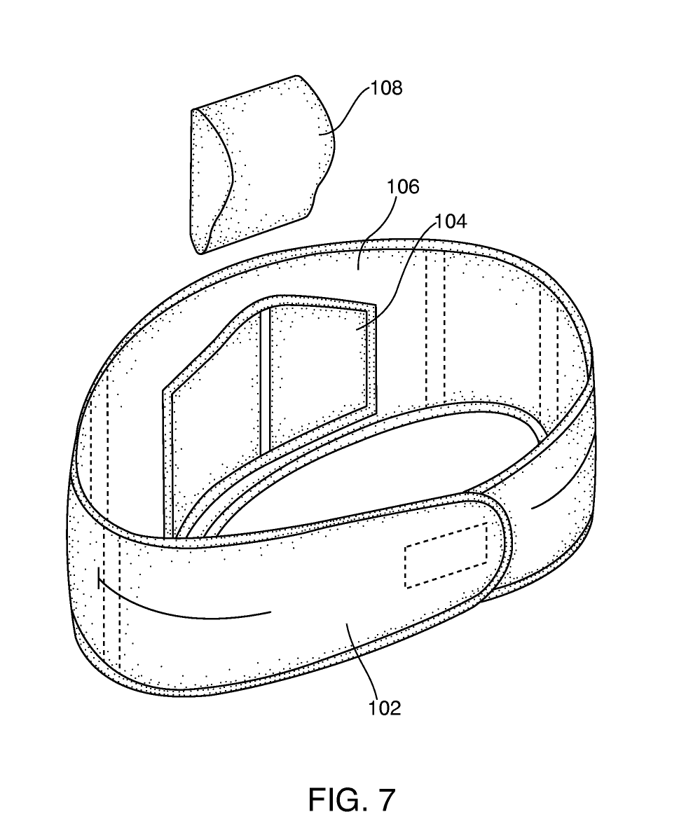

FIGS. 6 to 9 are directed to an embodiment of the invention wherein a orthotic is positioned in a belt-like member that can be positioned snugly around a user or wearer's waist. As shown, for example, in FIG. 6, a belt-like member 80 is positioned in the waist area 82 of a user or wearer 84. Belt-like member 80 has a dotted area 88 that represents any of the conventional closure means that could keep the respective ends 90 of belt-like member 80 snugly together. Such closure means include, but are not limited to, snaps, buttons, hooks, buckles, VELCRO sections, and the like. This is similarly applicable to the embodiments shown in FIGS. 7 to 9 as well. Belt-like member 80 may comprise, for example, a cloth-like fabric or material such as nylon, polyester, cotton, or vinyl.

In FIG. 7 a belt-like member 102 has a pouch 104 located on or in the rear inside surface 106 of belt-like member 102. Pouch 104 is configured to receive a orthotic 108.

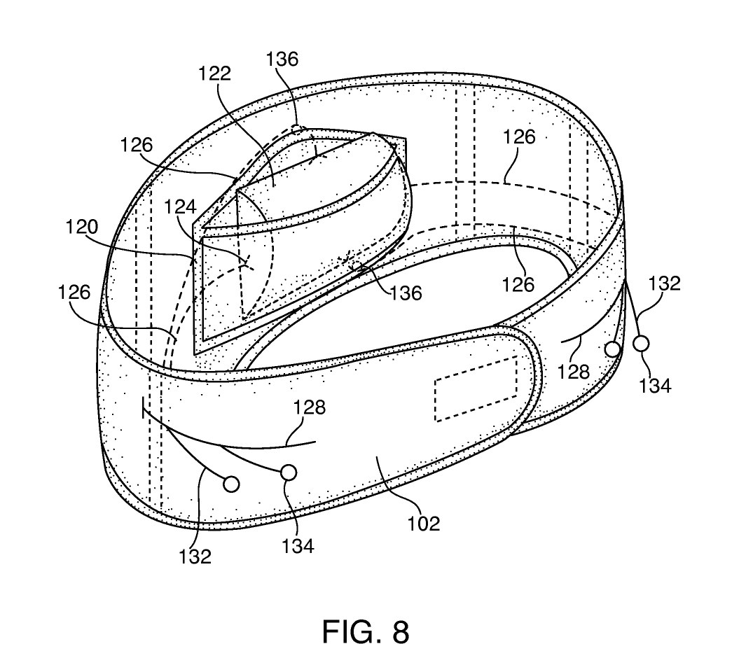

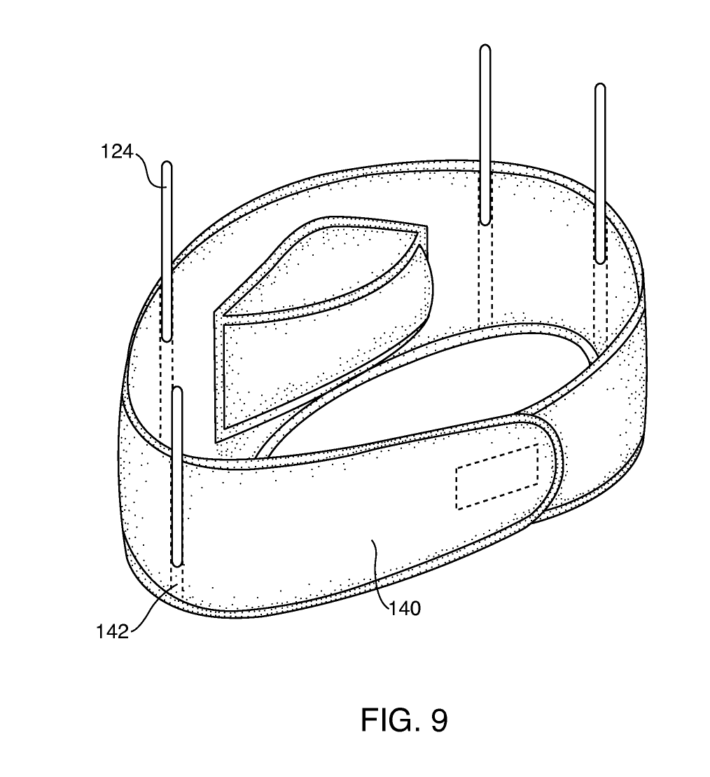

The embodiment of the invention set forth in FIG. 8 is a variation of the embodiment set forth in FIG. 7 where a belt-like member 118 has a pouch 120 that has received a orthotic 122. Pouch 120 is connected at connection points 124 to two or more wires 126 that extend through belt-like member 118 and exit at slits 128. The proximal ends 132 of wires 126 have rings or other gripping means 134 for the user (not shown) to adjust the position of orthotic 122 within pouch 120. With four wires 126 in place, the user should be able to adjust the vertical and horizontal position of orthotic 122 within pouch 120. The wires that adjust the vertical position wind around circular supports 136.

As shown in FIG. 9, a belt-like member 140 may have two more vertical chambers 142 that receive splines 144, to provide lateral support. Splines 144 are comprised of rigid or substantially rigid cleanable, wipable, or sterilizable material, such as plastic or a metal such as stainless steel.

The embodiments set forth in FIGS. 6 to 9 can be used separately, as an adjunct to separate pieces of radiation protective gear or garments. However, the belt-like members can also be integrated into or attached to radiation protective gear or garments.

Orthotics 108 and 122 comprise, for example, suitable polymeric foams or filling that is flexible but supportive. Such foams or fillers include, but are not limited to, polyvinyl, polyethylene, and polyurethane materials or gels. Optionally orthotic 108 or 122 could comprise an integral, battery operated heating, massaging, or vibrating device that could be operated by the wearer or a third party, dependent upon where the controls are positioned. The controls could be positioned on the front portion of a belt-like member so that the wearer could operate them or at the rear or side portion so that a third party could do so without disturbing the sterile field. Optionally the material used could retain heat or cold for a useful period of time, such that the orthotic would be heated or chilled just prior to usage.

The invention herein is intended for use with conventional and/or commercially available radiation protective gear or garments, such as ZEROGRAVITY or other similar radiation protective garments available from, for example, Burlington Medical Supplies of Newport News, Va. Such gear or garments could be used as is or modified to incorporate the lumbar support materials described herein. The materials to be used according to the invention include, for example, known polymeric materials that are cleanable, wipable, and/or in some cases sterilizable. The orthotics preferably comprise firm, substantially firm, resilient, and/or moldable supportive elastic materials, including gels or moldable pellets or other shapes that can be adapted to conform to a body's shape and that optionally retain heat or cold. It is within the scope of the invention that material such as neoprene, optionally with a stretch nylon laminate cover, could be used in different aspects of the invention. The useful radiation gear or garments preferably have seams to provide support to the different elements in the radiation gear or garments.

The materials and dimensions of the components of the various embodiments of the invention are fairly conventional. The orthotics typically may be from about 4 to about 14 in., preferably from about 6 to about 12 in., in width, from about 8 to about 16 in., preferably from about 10 to about 14 in., in height, and from about 1 to about 8 in. preferably from about 1.5 to about 6 in., in thickness, of varying shapes. The width of the supportive belt-like members would typically be from about 3 to about 12 in., preferably from about 4 to about 10 in., in width.

The terms “comprises”, “comprising”, “includes”, “including”, “having” and their conjugates mean “including but not limited to”. This term encompasses the terms “consisting of” and “consisting essentially of”.

The phrase “consisting essentially of” means that the composition or method may include additional ingredients and/or steps, but only if the additional ingredients and/or steps do not materially alter the basic and novel characteristics of the claimed composition or method.

As used herein, the singular form “a”, “an” and “the” include plural references unless the context clearly dictates otherwise. For example, the term “a compound” or “at least one compound” may include a plurality of compounds, including mixtures thereof.

The word “exemplary” is used herein to mean “serving as an example, instance or illustration”. Any embodiment described as “exemplary” is not necessarily to be construed as preferred or advantageous over other embodiments and/or to exclude the incorporation of features from other embodiments.

The word “optionally” is used herein to mean “is provided in some embodiments and not provided in other embodiments”. Any particular embodiment of the invention may include a plurality of “optional” features unless such features conflict.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

Although the present invention has been described in relation to particular embodiments thereof, many other variations and modifications and other uses will become apparent to those skilled in the art.

What is claimed is:

1. A lumbar support shown and described herein.

2. A lumbar support system as shown and described herein.

3. A system for modifying standard radiation protective gear or garments to provide internal lumbar support.

4. A system of claim 3, wherein the gear or garment is an apron, vest, skirt, jacket, smock, or other wearable garment or clothing design.

5. The system of claim 3, which comprises:
a mechanism of adapting the lumbar support to the inside or interior of the radiation protective gear or garment;
a mechanism for adjusting the support to the spinal configuration of a user or wearer; and
a mechanism for applying an external force via a fastening mechanism or an external belt system to enhance lumbar support.

6. A method of modifying standard radiation protective gear or garments, which comprises:
adapting lumbar support to the inside or interior of the radiation protective gear or garment;
providing a mechanism for adjusting the support to the spinal configuration of a user or wearer; and
providing a mechanism for applying an external force via a fastening mechanism or an external belt system to enhance lumbar support.
